SEQUENCE LISTING (1 sequence)

SEQ ID NO: 1 - protein, 5 aa, Artificial Sequence
  Description of Artificial Sequence L-m-sacrolysyl-L-arginyl-L-lysyl-L-m-sacrolysl-L- histidine methyl ester
XRKXH

(19) United States
(12) Patent Application Publication
(10) Pub. No.: US 2003/0162721 A1
(43) Pub. Date: Aug. 28, 2003

(54) PHARMACEUTICAL COMPOSITION CONTAINING PEPTICHEMIO

(76) Inventor: FRANCESCO MEHLEM, WORBLAUFEN (CH)

Correspondence Address: ANTHONY P VENTURINO, STEVENS DAVIS MILLER & MOSHER, 1615 L STREET NW, SUITE 850, WASHINGTON, DC, 20036

(21) Appl. No.: 09/462,155

(22) PCT Filed: Jul. 7, 1998
(86) PCT No.: PCT/CH98/00300

(30) Foreign Application Priority Data
Jul. 7, 1997 (CH) 1651/97

(51) Int. Cl. A61K 38/05; A61K 31/724

(52) U.S. Cl. 514/18; 514/58

(57) ABSTRACT

Pharmaceutical compositions are made available which serve to treat cancers, particularly melanomas. As active component the composition contains at least one peptide compound which contains L-m-sarcolysine as amino acid component and and which is selected from the following group:

-L-seryl-L-p-fluorophenylalanyl-L-m-sarcolysine

-L-prolyl-L-m-sarcolysyl-L-p-fluorophenylalanine

-L-m-sarcolysyl-N-nitro-L-arginyl-L-norvaline

-L-p-fluorophenylalanyl-L-m-sarcolysyl-L-asparagine

-L-p-fluorophenylalanyl-glycyl-L-m-sarcolysyl-norvaline

-L-m-sarcolysyl-L-arginyl-L-lysyl-L-m-sarcolysyl-L-histidine

and lower alkyl esters and/or acid addition salts thereof. As auxiliary or carrier substance the composition contains at least one optionally substituted cyclodextrin. A composition containing PSF as active substance and hydroxypropyl-β-cyclodextrin as carrier substance has a particularly good activity. The compositions intended for parenteral application contain hydroxypropyl-β-cyclodextrin, whereas the agents made up as capsules for oral administration contain α-, β-, or γ-cyclodextrin.

The present invention relates to a pharmaceutical composition in which pharmaceutically active peptides containing L-m-sarcolysine as the amino acid component are formulated. The active substances serve particularly for chemotherapy against cancers and are utilized specially for melanomas. A carrier substance on a cyclodextrin basis serves for delayed release of the active substances and is responsible for a sufficient bioavailability during a sufficiently long period of time.

A complex of six peptides containing m-L-sarcolysine has become known under the trade name “Peptichemio” (Insituto Sieroterapico Milanese S. Belfanti, Milan, Italy) for chemotherapy against cancer. It has been found that the activity of the individual peptides is different and that particularly one representative exhibits very high toxicity to melanoma cells. The peptides are a development which began with the product “Melphalan,” i.e., 4-[bis(2-chloroethyl)]-amino-L-phenylalanine. It has been found that this product has a cytostatic effect and can be utilized both for myeloma and for melanoma therapy. For the further development of the active substance, derivatives of the product have been prepared. This also resulted in L-m-sarcolysine, which has been further derived in that peptides have been prepared which contain the modified amino acid as a component. A combination of six such oligopeptides formed the active principle of the antitumor agent “Peptichemio.” The six peptides are the following:

-L-seryl-L-p-fluorophenylalanyl-L-m-sarcolysyl ethyl ester

-L-prolyl-L-m-sarcolysyl-L-p-fluorophenylalanine ethyl ester

-L-m-sarcolysyl-N-nitro-L-arginyl-L-norvaline ethyl ester

-L-p-fluorophenylalanyl-L-m-sarcolysyl-L-asparagine ethyl ester

-L-p-fluorophenylalanyl-glycyl-L-m-sarcolysyl-norvaline ethyl ester

-L-m-sarcolysyl-L-arginyl-L-lysyl-L-m-sarcolysyl-L-histidine methyl ester

It has been found that Peptichemio is less toxic to human lymphoplasts than m-L-sarcolysine alone. In addition to the lesser toxicity of Peptichemio, reduced formation of DNA cross-links has been noted. In contrast thereto, increased cytotoxicity of Peptichemio to human melanoma cell lines has been noted, in comparison to m-L-sarcolysine. Here the higher cytotoxicity was associated with greater DNA cross-linking. The comparative analysis of the six peptides showed differences in the cytotoxicity to melanoma cells. One of the six peptides showed considerably higher cytotoxicity in comparison with Peptichemio itself (R. Levenson, et al., Radiumhemmet, Karolinska Hospital, Stockholm, Sweden, Eur. J. Cancer Clin. Oncol.; 23: 6, 783-788, 1987). According to these studies, it has been found that the peptide L-propyl-m-sarcolysyl-L-p-fluorophenylalanine (PSF) was 35 times and 28 times, respectively, more toxic to RPMI 8322 melanoma cells than melphalan and m-sarcolysine, respectively. Similar differences between the active substances have also been found for other melanoma cell lines.

In order that the activity found in vitro may likewise be developed in vivo, the bioavailability of the active substance in the body must be adequate. There must be a high enough concentration for a sufficient period of time, i.e., the half-life of the active substance must be sufficient.

It is accordingly the task of the present invention to make available a pharmaceutical composition by means of which one or more of the six peptides can be administered in such a way that the bioavailability satisfies the requirements set.

It has been found that a combination of one or more of the six peptides with a substituted cyclodextrin compound as a carrier meets these requirements.

The subject of the present invention is accordingly the pharmaceutical composition defined in patent claim 1. As active substance, the composition contains at least one of the peptides selected from the group:

-L-seryl-L-p-fluorophenylalanyl-L-m-sarcolysine

-L-prolyl-L-m-sarcolysyl-L-p-fluorophenylalanine

-L-m-sarcolysyl-N-nitro-L-arginyl-L-norvaline

-L-p-fluorophenylalanyl-L-m-sarcolysyl-L-asparagine

-L-p-fluorophenylalanyl-glycyl-L-m-sarcolysyl-norvaline

-L-m-sarcolysyl-L-arginyl-L-lysyl-L-m-sarcolysyl-L-histidine

and their lower alkyl esters, particularly their ethyl and methyl esters and/or acid addition salts thereof.

The peptides are preferably present in the form of hydrochlorides or hydrobromides. Preferably, L-prolyl-m-sarcolysyl-L-p-fluorophenylalanine (PSF) is used. The cyclodextrin carrier substance, which serves to regulate the bioavailability, is preferably hydroxypropyl-β-cyclodextrin. This product is available in commerce and has been described by Pitha et al. in “Hydroxypropyl-β-cyclodextrin preparation and characterization,” International Journal of Pharmaceutics, 29; 73 to 83 (1986). Hydroxypropyl-β-cyclodextrin is available in commerce under the trade name “Incapsin” of the firm of Janssen. β-cyclodextrin is used only for the oral and topical areas. For parenteral administration, a substituted β-cyclodextrin, preferably hydroxypropyl-β-cyclodextrin, is used. The cyclodextrin compound forms a complex with the peptides and, in parenteral application, causes the active substance to be made available in sufficient concentration in the organism. Preferably PSF is used together with hydroxypropyl-β-cyclodextrin as the active substance, a molar ratio of PSF to hydroxypropyl-β-cyclodextrin in the range of from 1:1 to 1:10 being used. Typical examples of such ratios are 1:1, 1:2, 1:3. The active substance forms with the cyclodextrin compound a complex in which the peptide is stabilized. The half-lives of the active substance on the order of magnitude of 10 to 30 min. in a body fluid are increased through inclusion in a complex with cyclodextrin, a control being possible through the above-mentioned ratio. The complex can be better taken in and absorbed by the organism, the bioavailability being positively influenced. The ratio must be adapted to the illness of the patient and his state of health. The product may be presented as a solid product or as a concentrate which is used for the preparation of injectable solutions or infusions. If therapeutically necessary, the pharmaceutical formulation may also contain other active substances.

For oral formulations, the PSF is mixed with α- or -β- or γ-cyclodextrin. Here the ratio of peptide to cyclodextrin is, for example, 1:1 to 1:10, typically 1:1, 1:2, 1:3. The combination is, if need be, packed in capsules together with a suitable carrier substance.

### EXAMPLE OF PREPARATION (ACTIVE SUBSTANCE)

Synthesis of L-prolyl-L-m-sarcolysyl-L-p-fluorophenylalanine ethyl ester hydrochloride

a) N-carbobenzoxy-L-m-sarcolysyl-L-p-fluorophenylalanine ethyl ester

52.5 g of L-p-fluorophenylalanine ethyl ester hydrochloride are treated with 75 ml of Na₂CO₃ (sodium carbonate) saturated solution and 150 ml of CHCl₃. The mixture is shaken out, and the organic phase is separated and saved. The aqueous phase is shaken out a second time with 75 ml of CHCl₃. The combined chloroform extracts are mixed and washed once with water, and then separated from the aqueous phase and dried on anhydrous Na₂SO₄. The concentration of amino acid ester is determined by a titration with HClO₄ (perchloric acid). The yield corresponds approximately to the theoretical value; it is at 98%.

286.5 ml of a chloroform solution containing 0.1905 moles of L-p-fluorophenylalanine

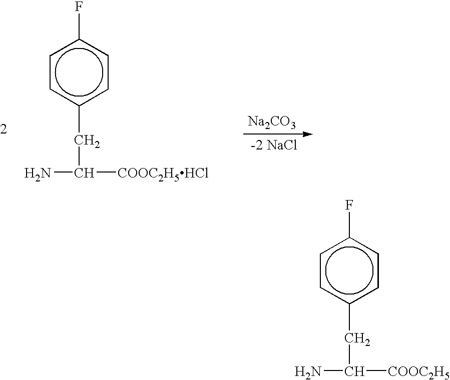

ethyl ester are reacted with 83.7 g (0.1905 moles) of N-cbzo-L-m-sarcolysine. The solution is cooled on an ice bath.

Added to the cooled solution with stirring are 41.25 g (0.200 moles of dicyclohexyl carbodiimide-DCC) and 60 ml of chloroform, the solution being constantly stirred with simultaneous cooling for 30 min. The mixture may possibly solidify into a solid mass. In this case, the mass is made liquid again through addition of 150 ml of chloroform, it being stirred with slight warming. In, this way, dissolving of the precipitated product is accelerated. The reaction is ended 2 hrs after addition of the DDC. The end of reaction is established by TLC checking (thin-layer chromatography; silica gel G layer, solvent: chloroform+acetone 9:1, manifestation by spraying with dilute, acid KMnO₄ solution). The precipitated dicyclohexyl urea is separated by filtration. The solution is washed first with little water, then with saturated Na₂CO₃ solution. The chloroform solution is shaken out once more with water and then dried with Na2SO4. The solvent is evaporated in vacuo and removed. After drying, 140.25 g of slightly yellowish-colored product is obtained (yield 98.3%). The substance produced has a melting point of 123-124.5° C. and is chromatographically homogeneous. Through crystallization of 4.5 g of substance from 37.5 ml ethyl alcohol, 3.75 g of a lighter product are produced with a melting point of 125-126° C. α_{D}²⁰:27.7 (c=2, CHCl₃).

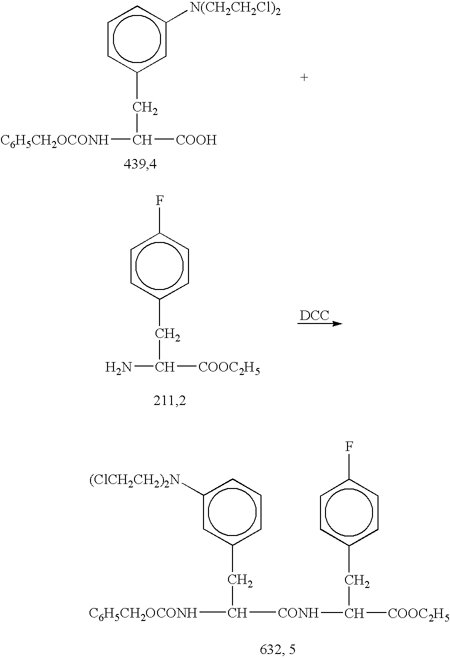

Analysis for C₃₂H₃₆Cl₂FN₃O₅

N %=6.67 (6.66 calculated

Cl %=11.5 (calculated=11.2)

b) L-m-sarcolysyl-L-p-fluorophenylalanine ethyl ester

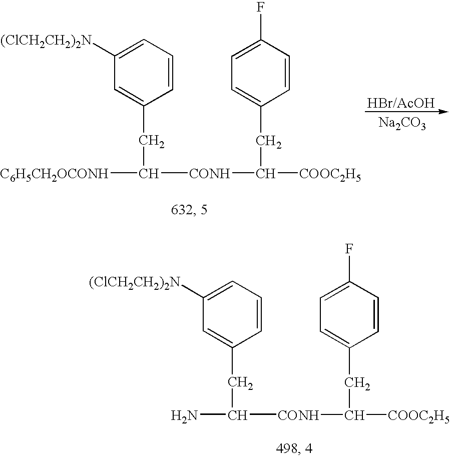

With exclusion of atmospheric humidity, 600 ml of HBr in glacial acetic acid (33%) are added with slow stirring to 390 g (0.616 moles) of die[?] M-carbobenzoxy-L-m-sarcolysyl-L-p-fluorophenylalanine ethyl ester. Dissolving and cessation of the CO2 development takes place after 40 minutes. It is allowed to stand for a further 20 minutes with stirring and diluted with app. 400 ml of ether. The whole is poured into 5 lt of ether which is kept under constant stirring, is decanted, and the precipitated oil is washed twice with 2 lt of ether with decanting. The oil is treated with 4 lt of water with stirring, and a solid is obtained which is collected after app. 30 min. by filtration and is completely washed with a total of 1500 ml of water and 500 ml of ether. The bromohydrate thus obtained is suspended in 2 lt of ethyl acetate and treated with stirring with 450 ml of saturated sodium carbonate solution, thus until the solution is alkaline. After dissolving has taken place, filtration is carried out on the suction filter in order to remove the suspended dicyclohexyl urea (very little). In a separating funnel, the organic layer is separated from the aqueous phase, and the aqueous phase is extracted with a further 500 ml of ethyl acetate. The purified extracts are washed with 300 ml of water, Na2CO4 dried, and treated with norite. Filtration is carried out, and the filtrate is dried in vacuo (40° C.). The residue is taken up even before it becomes firm in 500 to 1000 ml of ether. During the night, a white product is precipitated from the solution obtained. Yield: 247 g (80.4%) Melting point 100-102° C.

α_{D}²⁰=−7.50° (c=2, chloroform)

TLC (BuOH/AcOH/H₂O 65:15:25; KMnO₄ diluted):

one band, Rf=0.74

analysis for C₂₄H₃₀Cl₂FN₃O₃

N %=8.34 (8.43 calculated)

Cl %=14.1 (14.2 calculated)

c) N-carbobenzoxy-L-prolyl-L-m-sarcolysyl-L-p-fluorophenylalanine ethyl ester

A mixture of 249 g (0.5 moles) of L-m-sarcolysyl-L-p-fluorophenylalanine

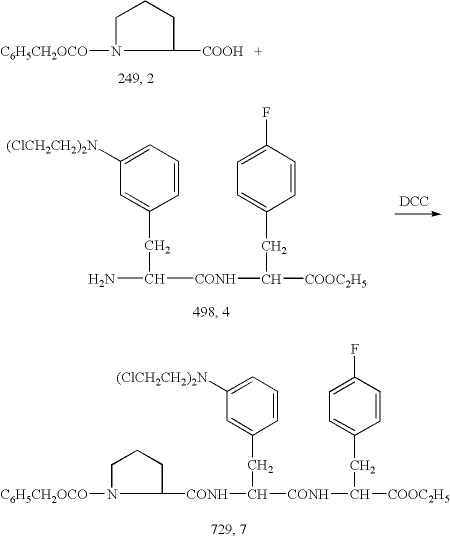

ethyl ester, 125 g (0.5 motes) of N-cbzo-L-proline, and 109 g (0.525 moles) DCC in 3000 ml of chloroform is allowed to stand for 30 minutes with stirring, with external cooling for a further 90 minutes at room temperature (TLC, silica gel G, Chf/Me₂CO 9:1; or with BuOH/AcOH/H₂O 65:15:25; KMnO₄, diluted, acid). After removal of the dicyclohexyl urea by filtration, the solvent is evaporated off in vacuo, and the residue, still in liquid state, is poured into 800 ml of ether. From the solution obtained, the product precipitates slowly out, which is collected on a filter. Yield 290 g (78.5%). Melting point=148-150° C., α_{D}²⁰=42.4° (c=2; chloroform)

Analysis for C₃₇H₄₃FCl₂N₄O₆

N %=7.78% (7.68 calculated)

Cl %=9.6 (9.7 calculated)

d) L-prolyl-L-m-sarcolysyl-L-p-fluorophenylalanine ethyl ester hydrochloride

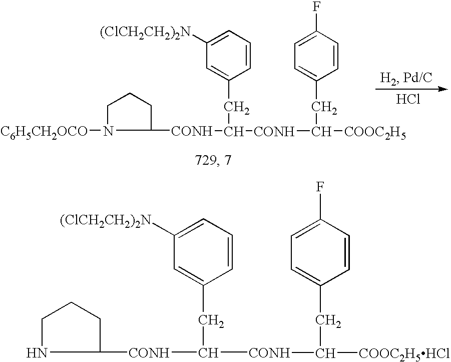

A mixture of 157.5 (0.261 moles) N-carbobenzoxy-L-prolyl-L-m-sarcolysyl-L-p-fluorophenylalanine ethyl ester and 30 g of palladium on 5% carbon is suspended under a stream of nitrogen in 15 ml of glacial acetic acid and 1750 ml of methanol. The reaction mixture is kept stirred and is reduced under a stream of hydrogen. After termination of the CO₂ development (after 4-5 hours), a TLC chromatography check is carried out (silica gel G), elution taking place with chloroform acetone 9:1 and making visible with dilute KMnO₄.

After removal of the catalyst by filtration, the filtrate is acidified with concentrated ethanolic HCl in a stoichiometric amount or a little more. The white, crystalline precipitate which slowly forms is collected on a filter and washed with ethanol or with ether: 85 g. The filtrate is concentrated practically to dryness, and the residue is recrystallized from ethanol: 25 g. Complete yield: 110 g (80.5%); melting point 122-124° C. (modification of the aggregate state)

α_{D}²⁰=13.0±0.5 (c=2; MeOH)

TLC (silica gel G; BuOh/AcOH/H₂O 65:15:25; KMnO₄ diluted: one band Rf=0.54.

Analysis for C₂₉H₃₈Cl₃FN₄O₄

N %=8.93% (8.86 calculated)

Cl %=16.7% (16.8 calculated)

Cl- % 5.65% (5.6 calculated)

### EXAMPLE 1

Parenteral Preparation for Treatment of Melanomas.

| | Component | Amount |
|---|---|---|
| Peptide | PSF ethyl ester.HCl | 8 g |
| Cyclodextrin-Carrier | Hydroxypropyl-β-cyclodextrin | 16 g |
| andere Wirk- und Hilfsstoffe | Chlophenamine | 32 mg |
| | Water, sterile | ad 100 ml |

| | |
|---|---|
| Form of administration: | sterile solution in ampule |
| Dosage unit: | 40 mg peptide/0.5 ml solution, |
| | 80 mg hydroxypropyl-β-cyclodextrin/0.5 |
| | ml, solution |
| | 1.6 mg chlorophenamine/0.5 ml solution |

Remarks: Intended for i.v. administration or for infusions.

### EXAMPLE 2

Oral Cytostatic in Capsules

| | Component | Menge (Dosage Unit) |
|---|---|---|
| Peptide | PSF-ethyl ester.HCl | 12 mg |
| Cyclodextrin carrier | β-Cyclodextrin | 25 g |

1. Pharmaceutical composition for the treatment of cancers containing as active component at least one peptide compound which is released with delay, characterized in that the peptide compound is selected from the group:
-L-seryl-L-p-fluorophenylalanyl-L-m-sarcolysine
-L-prolyl-L-m-sarcolysyl-L-p-fluorophenylalanine
-L-m-sarcolysyl-N-nitro-L-arginyl-L-norvaline
-L-p-fluorophenylalanyl-L-m-sarcolysyl-L-asparagine
-L-p-fluorophenylalanyl-glycyl-L-m-sarcolysyl-norvaline
-L-m-sarcolysyl-L-arginyl-L-lysyl-L-m-sarcolysyl-L-histidine
and lower alkyl esters and/or acid addition salts thereof, and that the composition contains at least one optionally substituted cyclodextrin as auxiliary or carrier substance.

2. Pharmaceutical composition according to claim 1, characterized in that the lower alkyl esters are methyl or ethyl esters.

3. Pharmaceutical composition according to claim 1 or 2 for parenteral application, characterized in that the optionally substituted cyclodextrin is hydroxypropyl-β-cyclodextrin.

4. Pharmaceutical composition according to one of the claims 1-3 for oral application, characterized in that the optionally substituted cyclodextrin is selected from α-, β-, and γ-cyclodextrin.

5. Pharmaceutical composition according to one of the claims 1-4, characterized in that the peptide compound is L-prolyl-L-m-sarcolysyl-L-p-fluorophenylalanine preferably the in the form of the hydrochloride of the ethyl ester.

6. Composition according to one of the claims 1-5, characterized in that the molar ratio of the peptide compound to the optionally substituted cyclodextrin is from 1:1 to 1:10 and preferably from 1:2 to 1:4.

7. Composition according to one of the claims 1-6, characterized in that additionally it further contains at least one pharmacologically active substance.

8. Composition according to claim 7, characterized in that the additional pharmacologically active substance is chlorophenamine.
